(19) United States
(12) Patent Application Publication
(10) Pub. No.: US 2024/0382937 A1
(43) Pub. Date: Nov. 21, 2024

(54) CATALYST FOR THE PARTIAL OXIDATION OF N-BUTANE TO MALEIC ANHYDRIDE

(71) Applicant: POLYNT S.P.A., SCANZOROSCIATE (IT)

(72) Inventors: Carlotta CORTELLI, Bologna (IT); Laura FRATALOCCHI, MILANO (IT); Lorenzo GRAZIA, BERGAMO (IT)

(73) Assignee: POLYNT S.P.A., SCANZOROSCIATE (IT)

(21) Appl. No.: 18/691,983

(22) PCT Filed: Jul. 4, 2022
(86) PCT No.: PCT/EP2022/068395

§ 371 Date: Mar. 14, 2024

(30) Foreign Application Priority Data
Sep. 14, 2021 (IT) 102021000023639

(51) Int. Cl. B01J 27/199; B01J 27/198; B01J 37/08; C07D 307/60

(52) U.S. Cl. CPC B01J 27/199; B01J 27/198; B01J 37/08; C07D 307/60

(57) ABSTRACT

The present invention relates to a vanadium and phosphorus mixed oxide (VPO) catalyst for the partial oxidation of n-butane to maleic anhydride. comprising vanadyl pyrophosphate as main component and at least one first promoter element selected from cobalt, iron, copper and mixtures thereof. The present invention further relates to a process for the production of maleic anhydride by partial oxidation of n-butane in the presence of the above mentioned catalyst.

The present invention relates to a catalyst for the partial oxidation of n-butane to maleic anhydride. The catalyst is characterized by high selectivity and an increased yield of maleic anhydride. The invention further relates to a process for the production of maleic anhydride in the presence of the above mentioned catalyst.

Maleic anhydride is a well-known and versatile intermediate for the production of unsaturated polyester resins, pharmaceutical products and agrochemical products. Initially, it was produced on an industrial scale by selective oxidation of benzene with catalysts based on oxides of vanadium/molybdenum. Nowadays, benzene has for the most part been replaced by non-aromatic hydrocarbons, in particular n-butane, as a starting raw material.

The process of selective oxidation of n-butane to maleic anhydride is conducted in the gaseous phase, in the presence of a vanadium and phosphorus mixed oxide catalyst (so-called “VPO” catalyst) which comprises vanadyl pyrophosphate of formula (VO)₂P₂O₇ as main component. On an industrial scale, the process is typically conducted at a conversion of n-butane in a range of 80-86%, with yields in weight of maleic anhydride of 96-103%. The main byproducts of the process are CO and CO₂(COₓ), but acetic acid and acrylic acid are also formed with yields in weight of 2.5-3%. Of these byproducts, acrylic acid is particularly undesired, in that it causes problems of corrosion and encrustations in the downstream section of industrial plants for producing maleic anhydride, resulting in a decrease of the final efficiency of purification.

Since n-butane has a low reactivity, oxidation is conducted at high temperatures, which places limits on the obtainable selectivity for maleic anhydride. Being an extremely exothermic reaction, the temperature profile of the catalytic bed is characterized by the presence of a hot spot, which can reach temperatures even 50-60° C. higher than that of the reactor cooling liquid. The presence of this hot spot not only decreases the selectivity to maleic anhydride owing to excessive oxidation, but also risks causing the loss of phosphorus from the catalyst, thus determining an unwanted increase in the catalytic activity toward total oxidation to COₓ.

In order to develop VPO catalysts that are capable of reaching an ever-increasing yield to maleic anhydride, various strategies have been adopted in an attempt to increase both the activity of the catalyst, expressed as conversion of n-butane, and the selectivity to maleic anhydride. However, most of these strategies have been found to be effective only in increasing the activity of the catalyst.

One strategy that is often used to improve catalytic performance is addition of an element (known as a doping agent) to the catalyst formulation, which acts as a promoter of activity and/or of selectivity. Such promoters can act both as structural promoters, favoring the formation of certain crystalline phases over others, or influencing the superficial acidity or the morphological properties of the catalyst, and also as electronic promoters, acting on the intrinsic activity of the catalytic sites.

Almost all the promoter elements described in the scientific literature are capable of improving the activity of the catalyst and/or its stability (understood as an increase of the average life of the catalyst), but with negligible effects on the selectivity to maleic anhydride [J. Catalysis 143(1993) 215-226; J. Nat. Gas Chem. 20 (2011) 635-638], and only a few metals, like bismuth and samarium, are capable of increasing the selectivity as well as the activity [Catal. Today 164 (2011) 341-346; App. Surf. Sci. 351(2015) 243-249].

The literature has also described the use of molybdenum as a promoter suitable for decreasing the yield of acrylic acid, but without identifying a definitive way of selectively decreasing the yield of acrylic acid that does not compromise the maleic anhydride yield or that does not require the reaction conditions to be altered. For example, U.S. Pat. No. 5,945,368describes a catalytic bed with a double-layer configuration, wherein a layer of the VPO catalyst promoted with Mo is arranged downstream of the reactor after a layer of traditional VPO catalyst; however, the maleic anhydride yield is not preserved. In U.S. Pat. No. 5,360,916 and U.S. Pat. No. 6,194,587, in order to maintain the yield of maleic anhydride unaltered, a system is described wherein the n-butane oxidation reaction is carried out in two separate steps, through the use of two reactors in series, where the gaseous stream in output from the first reactor is cooled and subsequently fed to the second reactor.

In light of the above, the aim of the present invention is to provide a VPO catalyst for the partial oxidation of n-butane to maleic anhydride with improved performance over the current generation of commercial VPO catalysts.

Within this aim, an object of the invention is to provide a VPO catalyst that is capable of achieving a higher yield of maleic anhydride than that of the current generation of VPO catalysts, by increasing the selectivity to maleic anhydride of the catalyst and, preferably, also its activity (understood as conversion of n-butane).

Another object of the invention is to provide a VPO catalyst that is capable of minimizing the formation of acrylic acid during the oxidation of n-butane, without compromising the yield of maleic anhydride.

Finally, another object of the invention is to provide a process for producing maleic anhydride with high yield and selectivity.

This aim and these and other objects which will become better apparent hereinafter are achieved by a vanadium and phosphorus mixed oxide (VPO) catalyst for the partial oxidation of n-butane to maleic anhydride, comprising vanadyl pyrophosphate (VO)₂P₂O₇ as main component and a first promoter element selected from the group consisting of cobalt, iron, copper, and mixtures thereof in an amount corresponding to an atomic ratio of vanadium to first promoter element comprised between 250:1 and 20:1.

The above aim and objects are further achieved by a process for the production of maleic anhydride by partial oxidation of n-butane in an oxygen-containing gas mixture in the presence of the VPO catalyst according to the invention.

Further characteristics and advantages of the invention will become better apparent from the detailed description that follows and from the accompanying drawings wherein:

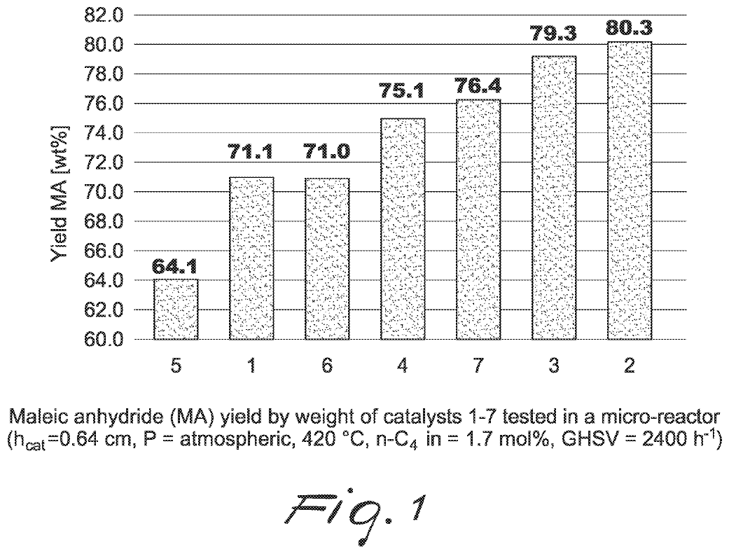

FIG. 1 is a chart showing the results in terms of yield of maleic anhydride obtained in the catalytic tests of Example 2 carried out in a micro-reactor; and

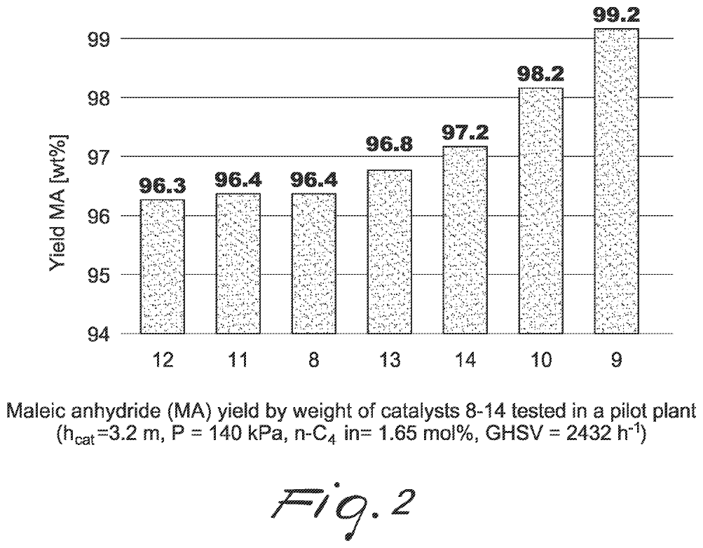

FIG. 2 is a chart showing the results in terms of yield of maleic anhydride obtained in the catalytic tests of Example 2 carried out in a pilot plant.

Following the research carried out by the inventors of the present invention, it has been possible to identify a narrow set of specific doping elements that, when added (individually or in a mixture) to the VPO catalyst to act as a promoter, are capable of increasing the selectivity to maleic anhydride of the catalyst and, preferably, also its activity (understood as conversion of n-butane), thereby increasing the yield of maleic anhydride.

Specifically, the VPO catalyst of the present invention comprises vanadyl pyrophosphate of formula ((VO)₂P₂O₇ as main component and a first promoter element selected from the group consisting of cobalt (Co), iron (Fe), copper (Cu), and mixtures thereof.

According to the invention, the above mentioned first promoter element is present in the catalyst in an amount corresponding to an atomic ratio of vanadium to first promoter element comprised between 250:1 and 20:1. The atomic ratio of vanadium to first promoter element can be comprised between 250:1 and 60:1, between 160:1 and 20:1, between 160:1 and 60:1, between 120:1 and 20:1, between 120:1 and 60:1, between 100:1 and 20:1, and between 100:1 and 60:1. Preferably, the atomic ratio of vanadium to first promoter element is 100:1.

In a preferred embodiment of the catalyst, the first promoter element is selected from the group consisting of cobalt, iron, and mixtures thereof.

In a more preferred embodiment, the first promoter element is cobalt.

In another more preferred embodiment, the first promoter element is iron.

Preferably, the catalyst according to the invention further comprises a second promoter element selected from bismuth and niobium. When present, the second promoter element is in an amount corresponding to an atomic ratio of vanadium to second promoter element comprised between 250:1 and 60:1.

In an embodiment, the second promoter element is niobium in an amount corresponding to an atomic ratio of vanadium to niobium comprised between 250:1 and 60:1, preferably equal to 160:1 or alternatively equal to 120:1. The VPO catalyst according to this embodiment is particularly suitable for performing the conversion of n-butane to maleic anhydride in a fluidized bed reactor.

In another embodiment, the second promoter element is bismuth in an amount corresponding to an atomic ratio of vanadium to bismuth comprised between 250:1 and 60:1, preferably 100:1. The VPO catalyst according to this embodiment is particularly suitable for performing the conversion of n-butane to maleic anhydride in a fixed bed reactor.

Advantageously, when the second promoter element is bismuth, the VPO catalyst of the invention can further comprise molybdenum as a third promoter element, in an amount corresponding to an atomic ratio of vanadium to molybdenum comprised between 250:1 and 60:1, preferably 100:1. The addition of molybdenum to the VPO catalyst of the invention in fact makes it possible to decrease the yield of acrylic acid (limiting the content of acrylic acid to amounts lower than 1 wt %), but without compromising the yield of maleic anhydride, and this without the need to use two different VPO catalysts in a double-layer configuration of the catalytic bed or separate reactors arranged in series.

In a preferred embodiment of the invention, the VPO catalyst comprises:
the first promoter element in an amount corresponding to an atomic ratio of vanadium to first promoter element of 100:1;
bismuth in an amount corresponding to an atomic ratio of vanadium to bismuth of 100:1; and
optionally molybdenum in an amount corresponding to an atomic ratio of vanadium to molybdenum of 100:1.

In the above mentioned preferred embodiment, the first promoter element is preferably selected from the group consisting of cobalt, iron, and mixtures thereof, and more preferably is cobalt or iron.

In another preferred embodiment of the invention, the VPO catalyst comprises:
the first promoter element in an amount corresponding to an atomic ratio of vanadium to first promoter element of 100:1; and
niobium in an amount corresponding to an atomic ratio of vanadium to niobium selected from 120:1 and 160:1.

Also in the above mentioned preferred embodiment, the first promoter element is preferably selected from the group consisting of cobalt, iron, and mixtures thereof, and more preferably is cobalt or iron.

In general, it has been observed that in VPO catalysts an atomic ratio of phosphorus to vanadium greater than 1 contributes to increase the activity of the vanadyl pyrophosphate and the selectivity to maleic anhydride. Therefore, in any of its embodiments described above, the VPO catalyst of the invention can have a phosphorus/vanadium (P/V) atomic ratio comprised between 1:1 and 1.8:1, preferably between 1.1:1 and 1.6:1.

The VPO catalyst of the present invention can be prepared according to methods known to the person skilled in the art, in which a thermal treatment (so-called “calcination”) of a precursor of the catalyst represented by a vanadyl acid orthophosphate hemihydrate formula (VO)HPO₄·0.5H₂O is performed.

The known methods for preparing the catalyst precursor (see for example U.S. Pat. No. 5,137,860 and EP 804963 A1) conventionally require the reduction of a pentavalent vanadium source (for example vanadium pentoxide V₂O₅ or suitable precursors such as for example ammonium metavanadate, vanadium chloride, vanadium oxychloride, vanadyl acetylacetonate, vanadium alkoxides) in conditions that lead the vanadium to a tetravalent state (average oxidation number +4), and the reaction of the tetravalent vanadium with a phosphorus source (for example orthophosphoric acid H₃PO₄). As a reducing agent, it is possible to use organic or inorganic compounds. Isobutyl alcohol is the most frequently used organic reducing agent is isobutyl alcohol, optionally mixed with benzyl alcohol.

In the preparation of promoted catalysts, each promoter element can be added in the form of a suitable precursor, for example of the acetylacetonate type or other commercially-known and used compounds or salts of the promoter element.

By way of example, the precursor of the VPO catalysts of the present invention can be prepared according to the method described in PCT publication WO 00/72963 publication. In accordance with this method, the vanadium source and the phosphorus source react in the presence of an organic reducing agent which comprises (a) isobutyl alcohol, optionally mixed with benzyl alcohol, and (b) a polyol, in a weight ratio (a): (b) comprised between 99:1 and 5:95.

The precursor is then filtered, washed and optionally dried, preferably at a temperature between 120° C. and 200° C.

After its preparation as above, the precursor may be subjected to pelletization, granulation and tableting.

The transformation of the precursor into the active VPO catalyst (calcination) entails the conversion of the vanadyl acid orthophosphate hemihydrate of formula (VO)HPO₄·0.5H₂O of the precursor into the vanadyl pyrophosphate of formula (VO)₂P₂O₇ of the active VPO catalyst. This transformation comprises heating the precursor in the presence of nitrogen, preferably up to a calcination temperature of less than 600° C., and maintaining it at said calcination temperature. Substantially all the calcination methods described in the art can be used, including a method in which the thermal treatment of the precursor comprises the following steps:
(a) optional initial heating of the precursor in air up to an initial temperature of 250-350° C.;
(b) optional holding of the initial temperature for 0.5-10 hours;
(c) heating of the precursor in nitrogen up to a calcination temperature of 500-600° C., and
(d) holding at said calcination temperature for 0.5-10 hours.

Once activated, the VPO catalyst is ready to be used in a process for the production of maleic anhydride according to the invention. According to such process, the production of maleic anhydride is carried out by partial oxidation of n-butane in a mixture with an oxygen-containing gas (for example air or oxygen) in the presence of the VPO catalyst of the invention according to any of its embodiments described above.

As a function of the geometry of the VPO catalyst, the reactor used in the process of the present invention can be of the fixed bed or fluidized bed type,. However, when the catalyst of the invention comprises bismuth as second promoter element, the reactor is preferably of the fixed bed type; alternatively, when the catalyst of the invention comprises niobium as second promoter element the reactor is preferably of the fluidized bed type.

The initial concentration of n-butane in the mixture with the oxygen- containing gas (i.e. the concentration of n-butane in the reactor feed) is generally comprised in a range from 1.00 to 4.30 mol %. The initial concentration of n-butane can be comprised between 1.00 and 2.40 mol %, preferably between 1.65 and 1.95 mol %, for example when the process is performed in a fixed bed reactor. Alternatively, the initial concentration of n-butane can be comprised between 2.50 and 4.30 mol %, for example when the process is performed in a fluidized bed reactor.

Preferably, the oxidation reaction is performed at a temperature from 320° C. to 500° C., preferably from 400° C. to 450° C.

The invention will now be described with reference to the following non-limiting examples.

### Example 1—Preparation of the Catalysts

Fourteen different VPO catalysts were prepared in order to carry out catalytic tests both in a micro-reactor (Table 1, catalysts 1-7), and in a pilot plant (Table 1, catalysts 8-14).

All the VPO catalysts were prepared as described below.

For the promoted catalysts, the first promoter element (PROMOTER I, P-I) was added in an amount corresponding to a constant atomic ratio of vanadium to promoter element equal to 100:1. The precursors (all of the acetylacetonate type) used to introduce the respective PROMOTER I into each catalyst are listed in Table 1.

The catalysts used for the tests in the pilot plant differ from those used for the tests in the micro-reactor due to the presence of bismuth as second promoter element (PROMOTER II, P-II). In particular, bismuth was introduced into catalysts 8-14 in an amount corresponding to an atomic ratio of V:Bi of 100:1, by adding during the synthesis, in the step of reduction of the vanadium source, the precursor Bi(C₈H₁₆O₂)₃ (bismuth 2-ethylhexanoate) having a titer of Bi equal to 24.6 wt % (170.6 g).

Synthesis and activation of the catalysts

All the syntheses of the VPO catalysts in Table 1 were carried out in a 30 L reaction flask, provided with heating jacket and reflux condenser, in which were placed 16.88 L of isobutyl alcohol, 1.815 L of benzyl alcohol, followed by the addition of 1834 g of vanadium pentoxide (V₂O₅), 2846 g of phosphoric acid (H₃PO₄ 100%) and, if applicable, the precursors of PROMOTER I and of PROMOTER II.

The reaction was conducted at approximately 106-110° C., keeping the system in total reflux for approximately 8 hours. At the end of the reaction, a product with the bright blue color of the precursorvanadyl acid orthophosphate hemihydrate of formula (VO)HPO₄·0.5H₂O was obtained. This product was removed from the flask and filtered through a Büchner funnel for approximately 6 hours. The solid residue (cake) resulting from filtration was placed in a tray and dried at ambient temperature for 24 hours. The material was then subjected to further drying at 150° C. for 8 hours and then precalcined at 220° C. for 3 hours and at 260° C. for 3 hours in an oven in static air.

The precalcined material thus obtained was mixed with 4% graphite and tableted in the form of small hollow cylinders (OD=4.8 mm, ID=1.7mm, L=4.7 mm).

The precalcined and tableted material was finally transformed into the active VPO catalyst by way of a final thermal treatment conducted in an oven, in a mixture of air, steam and nitrogen at 420° C. (ramp up rate equal to 2.5° C./min).

With the activation step concluded, the tablets of catalyst were used in the tests in the pilot plant, while for the tests in the micro-reactor the tablets were ground again in order to obtain the catalyst in the form of a fine powder.

TABLE 1

| | | Precursor | [g] | [wt %] | |
|---|---|---|---|---|---|
| Cat. | P-I | P-I | P-I | P-I | P-II |
| 1 | — | — | — | — | — |
| 2 | Co | C₁₅H₂₁Co(III)O₆ | 73.3 | 0.34 | — |
| 3 | Fe | C₁₅H₂₁Fe(III)I₆ | 73.4 | 0.32 | — |
| 4 | Mo | C₁₀H₁₄Mo(VI)O₆ | 66.4 | 0.56 | — |
| 5 | Mn | C₁₅H₂₁Mn(III)O₆ | 72.5 | 0.32 | — |
| 6 | Ni | C₁₀H₁₄Ni(II)O₆ | 54.5 | 0.34 | — |
| 7 | Cu | C₁₀H₁₄Cu(II)O₆ | 54.4 | 0.37 | — |
| 8 | — | — | — | — | Bi |
| 9 | Co | C₁₅H₂₁Co(III)O₆ | 73.3 | 0.34 | Bi |
| 10 | Fe | C₁₅H₂₁Fe(III)I₆ | 73.4 | 0.32 | Bi |
| 11 | Mo | C₁₀H₁₄Mo(VI)O₆ | 66.4 | 0.56 | Bi |
| 12 | Mn | C₁₅H₂₁Mn(III)O₆ | 72.5 | 0.32 | Bi |
| 13 | Ni | C₁₀H₁₄Ni(II)O₆ | 54.5 | 0.34 | Bi |
| 14 | Cu | C₁₀H₁₄Cu(II)O₆ | 54.4 | 0.37 | Bi |

Catalysts 1 and 8, without PROMOTER I, are not part of the invention and are used here as a reference standard, in order to compare the performance of the catalysts of the invention with those of the current generation of VPO catalysts. Catalysts 2, 3, 7, 9, 10 and 14, which comprise a PROMOTER I selected from Co, Fe and Cu, are part of the present invention. Finally, catalysts 4, 5, 6, 11, 12 and 13, which comprise a PROMOTER I selected from Mo, Mn and Ni, are not part of the invention.

Chemical/physical characteristics of the activated catalysts

For the reference catalysts which do not have PROMOTER I, a valency of 4.10 and a surface area of approximately 21 m²/g was found. The content of phosphorus and vanadium is in line with theoretical values, respectively 19 and 30 wt %, and the P/V ratio is equal to 1.05.

For the catalysts promoted with PROMOTER I, the valency is comprised in the range of 4.14-4.25 and the surface area in the range of 18-21 m²/g. It was observed that the most oxidized catalysts (higher valency) have a slightly lower surface area: Fe (4.23 and 19 m²/g), Mn (4.21 and 18 m²/g) and Ni (4.25 and 18 m²/g) compared to Co (4.14 and 21 m²/g), Cu (4.18 and 21 m²/g) and Mo (4.14 and 20 m2/g). The amount of P and V and the final P/V ratio are all in line with the values of the reference catalysts.

The main crystalline phase identified in all the activated catalysts is that of vanadyl pyrophosphate (VPP) of formula (VO)₂P₂0₇. In all the catalysts, the co-presence of the VPP phase and of VOPO₄phases was observed. These latter phases differ as a function of PROMOTER I. The δ-VOPO₄ phase, which is not active in the n-butane oxidation reaction, but which is the most selective for maleic anhydride, was clearly distinguishable in the activated catalysts promoted with Co, Fe and Cu, and present only in trace amounts in the catalysts promoted with Mo, Mn and Ni. In all the activated catalysts, the presence of the VOPO₄·2H₂O phase was also observed, except for the catalyst promoted with Mn. The presence of the VOPO₄·2H₂O phase is particularly desirable, since its conversion to δ-VOPO₄ appears to be favored under the reaction conditions. The presence of the inactive β-VOPO₄ phase was clearly visible only in the reference catalysts. In the catalysts promoted with Co, Fe, Cu and Ni, the presence of trace amounts of αII-VOPO₄, a phase that is known to bring benefits to the reaction in terms of activity (not of selectivity) only if present in trace amounts, was also observed.

The VPO catalysts used here in the pilot scale tests were re-analyzed after unloading from the reactor. In all the unloaded samples, a sharp decrease in the valency was noted when compared to the corresponding fresh catalysts, going from the range of 4.10-4.25 to 4.02-4.05. The inventors of the invention believe that this may be attributed to a change in the crystalline phases present in the activated catalyst which occurred during the reaction of the mixture of n-butane and air at high temperature.

The most abundant phase of vanadium in all the catalysts unloaded from the pilot plants was found to be the phase consisting of VPP and VO(PO₃)₂. In the reference catalyst without PROMOTER I and in the samples promoted with Mn and Ni, an abundant presence of the αII-VOPO₄ phase was further noted.

### EXAMPLE 2—CATALYTIC TESTS

Study of the catalytic performance was conducted both on a laboratory scale, in a micro-reactor, with samples of catalysts 1-7 in powder form tested at atmospheric pressure and in the absence of diffusive limitations of mass and heat, and in a pilot-scale fixed bed plant, with pelleted samples of cylindrical form of catalysts 8-14, tested under operating conditions applicable at industrial level.

Setup of the tests in a micro-reactor

The catalytic performance of VPO catalysts 1-7 of Table 1 were studied in a micro-reactor with an inner diameter (ID) of 1.4 cm inserted into an electric resistance oven under the following reference operating conditions:

| Pressure= | atmospheric; | |
|---|---|---|
| Reaction temperature= | 420° | C.; |
| Inlet n-butane concentration= | 1.70 | mol %; |
| Gas hourly space velocity (GHSV)= | 2400 | h⁻¹. |

The amount of each catalyst used for the respective test was 0.8 g, corresponding to a height of the catalytic bed equal to 0.64 cm. The thermocouple for controlling the reaction temperature was placed at the center (≈0.32 cm), inside the catalytic bed,.

Once the micro-reactor is loaded, the catalyst was equilibrated for approximately 50 hours at 400° C., under the same conditions of n-butane and air used during the reaction.

The composition of the reaction products in the gaseous phase was analyzed by gas chromatography.

Results of the tests in a micro-reactor

In all the reactivity tests, the reaction temperature was kept constant and equal to 420° C., thus making it possible to compare the results in terms of both conversion of n-butane (n-C₄) and selectivity to the main reaction products, i.e. maleic anhydride (MA), COₓ, acetic acid and acrylic acid. The results are shown in Table 2 below and in graphic form in FIG. 1.

TABLE 2

| | Conv. n-C₄ | Sel. MA | Sel. COₓ | Acetic acid | Acrylic acid |
|---|---|---|---|---|---|
| Cat. | [%] | [mol %] | [mol %] | yield [wt %] | yield [wt %] |
| 1 | 68.2 | 61.8 | 35.9 | 1.7 | 2.1 |
| 2 | 72 | 66.1 | 31.4 | 1.8 | 2.2 |
| 3 | 72.1 | 65.2 | 32.3 | 1.8 | 2.2 |
| 4 | 73.5 | 61.6 | 37.2 | 1.2 | 0.6 |
| 5 | 65.5 | 58.0 | 40.0 | 1.4 | 2.1 |
| 6 | 67.9 | 62.0 | 36.2 | 1.8 | 2.1 |
| 7 | 69 | 64.5 | 33.2 | 1.6 | 2.2 |

At a temperature of 420° C., catalyst 1 (non-promoted reference standard) reached 68.2% of conversion of n-butane and showed a selectivity to maleic anhydride of 61.8%, from which derives a yield by weight of maleic anhydride equal to 71.1 wt %.

Catalyst 5 (promoted with Mn) showed worse catalytic performance than all the catalysts tested, in terms of both activity and selectivity to maleic anhydride, in particular in view of the fall in n-butane conversion.

Catalyst 6 (promoted with Ni) showed catalytic performance levels that are practically similar to those obtained with the reference catalyst. The catalysts characterized by the best catalytic performances in terms of yield of MA were those promoted with Co (cat. 2), Fe (cat. 3), Cu (cat. 7) and Mo (cat. 4). The presence of cobalt, iron or copper resulted in an improvement of catalytic performance in terms of both conversion of n-C4and selectivity to MA, as can be seen from the data in Table 2. The effect on selectivity to MA is particularly surprising, in that the present inventors are not aware of such an effect having been previously described in the scientific and patent literature.

In the case of the catalyst promoted with molybdenum, the improvement of the yield of MA was mainly due to an increase in the conversion of n-C4. The selectivity to maleic anhydride in fact remained almost constant and equal to that of the standard sample.

An examination of the data for the reaction byproducts shows that the yields of acids are similar for all the catalysts and are comprised between 2.0-2.5 wt %, with the exception of catalyst 4 promoted with Mo, for which there is a sharp decrease in the yield of acrylic acid.

This is in line with the literature (for example U.S. Pat. No. 5,945,368) regarding the effect of Mo in decreasing the formation of acrylic acid. However, differently from what is already known, the present inventors have observed that the catalyst promoted with Mo according to their synthesis has been found to be effective in decreasing acrylic acid while at the same time preserving selectivity to maleic anhydride, without the need to abandon a single-layer configuration of the catalytic bed.

Setup of the tests in a pilot plant

The catalytic performance of VPO catalysts 8-14 of Table 1 were studied on a pilot scale in a jacketed fixed bed reactor, loaded with a catalytic bed with a height of 3.2 m, corresponding to approximately 850 g of catalyst. The inner diameter of the reactor is 2.1 cm. The reaction temperature was controlled by a thermocouple arranged inside a sheath, which in turn was placed inside the catalytic bed.

Once the reactor was loaded, the same startup procedure was carried out for all the catalytic tests. In particular, a mixture of air and 1.1 mol % of n-C4 was fed at a gas hourly space velocity (GHSV) of 1981 h⁻¹, up to a temperature of 340° C., with a ramp-up rate of 20° C./hour for 24 hours, at a pressure of 90 kPa (0.9 barg). Subsequently, the GHSV was adjusted to a value of 2200 h⁻¹, with a concentration of n-C₄ of 1.5 mol %, at a temperature of 380° C. with a ramp-up rate of 10° C./hour for a further 24 hours, at a pressure of 140 kPa (1.4 barg). Finally, the GHSV was brought to the setpoint value of 2432 h⁻¹, with a concentration of n-C₄ of 1.65 mol % and a constant pressure of 140 kPa (1.4 barg). The temperature of the salt bath was then adjusted to reach the n-C4 conversion value of 81.5%.

The catalytic tests were then conducted, maintaining the above mentioned salt bath temperature and with the further following reference operating conditions:

| | | |
|---|---|---|
| Air flow= | 2650 | Nl/h; |
| Inlet n-butane concentration= | ~1.64 | mol %; |
| Gas hourly space velocity (GHSV)= | 2432 | h⁻¹; |
| Pressure at entry= | 140 | kPa. |

The non-condensable reaction products were analyzed continuously via in-line gas chromatography, while the condensable products were absorbed in an aqueous solution and subsequently sampled in an external gas-mass device.

Results of the tests in a pilot reactor

In all the reactivity tests, the salt bath temperature (SBT) was adjusted so as to reach an n-C₄ conversion of approximately 81.5%. The comparison between the catalysts was carried out for the same lifespan of the catalysts (approximately 700 hours), so as to exclude effects deriving from possible deactivation phenomena. The results are shown in Table 3 below and in graphic form in FIG. 2.

TABLE 3

| | SBT | Sel. MA | | Acetic acid | Acrylic acid |
|---|---|---|---|---|---|
| Cat. | [° C.] | [mol %] | CO/CO₂ | yield [wt %] | yield [wt %] |
| 8 | 410 | 70.1 | 1.31 | 1.9 | 2.3 |
| 9 | 406 | 72.1 | 1.32 | 1.6 | 2.2 |
| 10 | 407 | 71.4 | 1.32 | 1.5 | 2.0 |
| 11 | 405 | 70.2 | 1.34 | 1.4 | 0.7 |
| 12 | 416 | 70.0 | 1.36 | 1.4 | 2.1 |
| 13 | 410 | 70.5 | 1.39 | 1.7 | 2.3 |
| 14 | 411 | 70.8 | 1.36 | 1.8 | 2.5 |

Since the oxidation reaction of n-C₄ is exothermic, a greater activity of the catalyst corresponds to a lower temperature of the cooling salt bath, at which temperature a determined value of n-C₄ conversion is reached. Therefore, in the case under examination the most active catalysts are those that reached the n-C₄ conversion value of 81.5% at the lower SBT.

As shown in Table 3, catalyst 8 (non-promoted reference standard) reached the value of 81.5% of n-C₄ conversion at the SBT of 410° C., and at that temperature it showed a selectivity to maleic anhydride of 70.1 mol %, from which it follows a yield by weight of maleic anhydride equal to 96.4wt %. With regard to the byproducts, catalyst 8 showed a CO/CO₂ ratio of 1.31, a yield of acetic acid of 1.9 wt % and a yield of acrylic acid of 2.3 wt %.

Catalyst 9 (promoted with Co) was the best-performing, showing both a high activity (lower SBT), and a high selectivity to maleic anhydride in comparison to all the catalysts in the test, reaching a yield by weight of maleic anhydride equal to 99.2 wt %.

Catalyst 10 (promoted with Fe) also showed a higher selectivity to maleic anhydride and higher activity compared to the standard catalyst 8,reaching a yield of maleic anhydride of 98.2 wt %.

Although the effect is less marked than the catalysts promoted with Co and Fe, catalyst 14 (promoted with Cu) also achieved an improvement of selectivity to maleic anhydride with respect to the reference catalyst, thus reaching a higher yield by weight of maleic anhydride (97.2 wt %). By contrast, no effects were noted in terms of activity, since the recorded SBT was in fact similar to that of the reference catalyst 8.

With regard to the formation of byproducts, the catalysts promoted with Co, Fe and Cu showed CO/CO₂ ratios and acid yields similar to those of the reference catalyst.

Catalyst 11 (promoted with Mo) reached the desired conversion of n-C₄ at the temperature of 405° C., showing a high activity, but a selectivity to maleic anhydride that was unchanged compared to the reference catalyst, thus resulting in a yield by weight of maleic anhydride equal to that obtained with catalyst 8. Differently from all the other catalysts, adding Mo to the formulation of the catalyst produced a sharp decrease, equal to approximately 70%, of the content of acrylic acid produced compared to all the catalysts tested.

Unpromising results were obtained both from catalyst 12 (promoted with Mn) and from catalyst 13 (promoted with Ni). In particular, the effect of adding Mn was to worsen performance with respect to the reference catalyst, since catalyst 12 reached 81.5% of conversion of n-C₄ at a higher salt bath temperature compared to catalyst 8, while the effect of adding Ni was almost negligible, since catalyst 13 showed performance that are entirely similar to the reference catalyst.

### Conclusions

On analyzing the data obtained in the micro-reactor and in the pilot plant, the present inventors observed a good correlation between the two data sets, both in terms of activity (based on a comparison of the trend of n-C₄ conversion values on a laboratory scale and the trend of salt bath temperature values on a pilot scale), and in terms of selectivity to maleic anhydride of the catalysts that were tested.

The catalysts that were found to be most selective to maleic anhydride on a laboratory scale were the catalysts promoted with at least one of cobalt, iron or copper, and these are the same catalysts that in the tests on a pilot scale, under industrial conditions, ensured the highest yield of maleic anhydride.

Similarly, for the catalysts that showed the worst catalytic performance (the catalysts promoted with Mn or Ni) on a laboratory scale, a low yield of maleic anhydride was also observed on a pilot scale.

Finally, although the addition of Mo did not increase catalytic performance, the beneficial effect of that element in decreasing the formation of acrylic acid, while at the same time maintaining selectivity to maleic anhydride unaltered when compared to the reference catalyst, was clear both in the micro-reactor and in the pilot plant.

The deviations obtained in absolute value between the results of the two test configurations should not be considered significant, as they can be attributed to different operating conditions (plant pressure), to the presence of hot spots along the 3.2 m catalytic bed used in the pilot plant, and/or to the different form/dimension of the catalyst. In fact, with reference to this last aspect, it should be noted that the use of a catalyst in powder form, compared to the form of cylindrical pellets, can lead to the creation of limitations on the diffusion of mass and heat, which partially influence catalytic performance.

In practice it has been found that the catalyst according to the invention fully achieves the set aim, in that it provides a catalytic system for the partial oxidation of n-butane to maleic anhydride that—with respect to the current generation of commercial VPO catalysts—is characterized by an improvement in catalytic performance in terms of increase in the yield of the product of interest, by virtue of an increased selectivity to maleic anhydride or of a simultaneous increase in selectivity and in activity (expressed as conversion of n-butane).

Furthermore, it has been observed that the catalyst according to the invention, in its embodiments in which molybdenum is present as an additional promoter element, also achieves the aim of minimizing the formation of acrylic acid, without compromising the yield of maleic anhydride.

Finally, it has also been observed that the present invention fulfills the object of providing a process for producing maleic anhydride with high yield and selectivity.

The disclosures in Italian Patent Application No. 102021000023639 from which this application claims priority are incorporated herein by reference.

1-10. (canceled)

11. A vanadium and phosphorus mixed oxide (VPO) catalyst for the partial oxidation of n-butane to maleic anhydride, comprising vanadyl pyrophosphate (VO)₂P₂O₇ as main component and a first promoter element selected from the group consisting of cobalt, iron, copper, and mixtures thereof in an amount corresponding to an atomic ratio of vanadium to first promoter element comprised between 250:1 and 20:1.

12. The catalyst according to claim 11, wherein the first promoter element is selected from the group consisting of cobalt, iron, and mixtures thereof.

13. The catalyst according to claim 11, further comprising a second promoter element selected from bismuth and niobium in an amount corresponding to an atomic ratio of vanadium to second promoter element comprised between 250:1 and 60:1.

14. The catalyst according to claim 13, wherein the second promoter element is niobium.

15. The catalyst according to claim 13, wherein the second promoter element is bismuth.

16. The catalyst according to claim 15, further comprising molybdenum as a third promoter element.

17. The catalyst according to claim 11, wherein:
the first promoter element is in an amount corresponding to an atomic ratio of vanadium to first promoter element of 100:1;
the catalyst optionally comprises a second promoter element selected from bismuth and niobium in an amount corresponding to an atomic ratio of vanadium to second promoter element of 100:1 when the second promoter element is bismuth, and in an amount corresponding to an atomic ratio of vanadium to second promoter element selected from 120:1 and 160:1 when the second promoter element is niobium; and
the catalyst optionally comprises molybdenum as a third promoter element, in an amount corresponding to an atomic ratio of vanadium to third promoter element of 100:1, when the second promoter element is bismuth.

18. The catalyst according to claim 15, comprising:
the first promoter element in an amount corresponding to an atomic ratio of vanadium to first promoter element of 100:1;
bismuth in an amount corresponding to an atomic ratio of vanadium to bismuth of 100:1; and
optionally molybdenum in an amount corresponding to an atomic ratio of vanadium to molybdenum of 100:1.

19. The catalyst according to claim 11, having a phosphorus/vanadium (P/V) atomic ratio comprised between 1:1 and 1.8:1, preferably between 1.1:1 and 1.6:1.

20. A process for the production of maleic anhydride by partial oxidation of n-butane in an oxygen-containing gas mixture in the presence of a catalyst according to claim 11.
